(19) United States
(12) Patent Application Publication
(10) Pub. No.: US 2008/0262101 A1
(43) Pub. Date: Oct. 23, 2008

(54) Method to Prepare a Personal Care Composition From a Base Composition and a Method to Prepare the Base Composition

(76) Inventors: Maria Teresa Belmar, Sharnbrook (GB); Quynh Pham, Berkeley Heights, NJ (US); Jason Richard Stokes, Sharnbrook (GB); Julia Helen Telford, Sharnbrook (GB); Shiping Zhu, Sharnbrook (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/662,243

(22) PCT Filed: Sep. 6, 2005
(86) PCT No.: PCT/EP05/09572

§ 371 Date: Mar. 11, 2008

(30) Foreign Application Priority Data
Sep. 9, 2004 (EP) 04255479.0

(51) Int. Cl. A61K 8/34; A61K 8/37; A61K 8/39; A61K 8/92

(52) U.S. Cl. 514/772.3; 514/785; 514/772

(57) ABSTRACT

A base composition is provided which comprises water and a non-ionic surfactant mixture. The base composition is suitable for use in preparing a personal care product.

### FIELD OF THE INVENTION

The invention relates to a process for preparing a personal care product such as a cream or lotion, from a base composition, wherein the process enables the production of a large variety of products in an efficient way.

### BACKGROUND

Personal care compositions such as skin creams and lotions are typically prepared on an industrial scale in a centralised manner, using large-scale equipment. In such a typical production process, for the production of products which are typically emulsions, the highest melting point ingredients are typically heated in a large container until they are molten (typically at around 80° C.), stirred, and further ingredients are added to the composition as it is continually stirred and cooled. The last of these ingredients is often perfume, which may be added when the composition is typically at around 40° C. Thereafter, the composition is cooled and dosed into retail containers. The time and speed of shearing used in the process typically effects the droplet size of the resulting emulsion.

A number of problems may be associated with the common large-scale methods of manufacture. Foremost of these is the difficulty of individualization or customization of products, with the result that typically only relatively few products may be provided to the consumer. Preparation of customized products by changing a minor feature e.g. color, fragrance, presence/amount of promotionals or minor components such as vitamins and retinol is however a desire that exists among users of skin creams and lotions.

A disadvantage of the known production process for producing skin creams and lotions is the time and cost associated with changing products on a production line; because of the need to thoroughly clean the production apparatus between production runs of different products, especially with large scale manufacturing equipment, this contributes significantly to the difficulties of producing many different products on such a line within a reasonable time scale.

As a consequence of this, where a product is a customized or individualized product, and the batch of that particular product is relatively small, the cost of the product is relatively high.

In addition, a typical skin cream composition may easily comprise 60-90% water. As such, water makes up a large proportion of the weight of a topical product. It therefore represents a large proportion of the cost of transporting such a commercial product between manufacture and retailing. Further more, the cost for heating and cooling the water in the preparation is also high and the processing time is usually long, 3-5 hrs.

As a consequence of the complicated known production process only a limited variety of products are made by industrial producers of personal care products which rely on central manufacturing of their products.

FR-A-2649318 discloses the use of a pre-emulsion to overcome instability problems with certain ingredients and physical instability of the emulsion. The pre-emulsion is prepared from a final oil in water emulsion which is dehydrated. The resulting stock called pre-emulsion is reconstituted with water to form the final skin care composition. A disadvantage of this process is the high cost associated with forming an emulsion and then de-hydrating it.

DE 3306943 discloses a process wherein a mono di or tri stearate and Tween™ are spray dried. Water is added to the spray dried composition to obtain a cream. This document does not disclose a process for preparing a personal care composition with a large variety in composition.

Even if there is variety presented in the prior art compositions, it is often only in certain aspects e.g. color, fragrance and minor ingredients because these may be post-added to a finished base product.

Usually texture of the cream, feel on skin and moisturizing properties can not be varied.

It is an object of the current invention to overcome this drawback of central manufacturing of personal care products.

### SUMMARY OF THE INVENTION

We have now found that it is possible to provide a wide variety of products when starting from a specific non-ionic base composition and treating that in a few carefully selected steps.

Therefore the invention relates to a process for the preparation of a personal care composition, which process comprises the steps of
a) Providing at a temperature of from 60 to 150° C., a base composition comprising 1 to 90 wt % water and a non-ionic surfactant mixture which comprises a first non-ionic surfactant selected from the group consisting of long chain fatty alcohols and long chain fatty acid mono esters of ethylene glycol, -glycerin, -sorbitol or a combination thereof and a second non-ionic surfactant, selected from the group consisting of polyethylene glycol esters of long chain fatty acid, polyethylene glycol ethers of long chain fatty alcohol, or a mixture thereof, wherein the molar ratio of the first non-ionic surfactant to the second non-ionic surfactant is from 10 to 1 to 80 to 1;
b) Cooling the composition obtained in step (a) to a temperature below 60° C., preferably to a temperature from 5 to 30° C., more preferred from 10 to 25° C.,
c) diluting the base composition until a surfactant concentration between 8 wt % and 30 wt % is obtained;
d) setting the sensory perception and the functionality of the composition by adding at least one ingredient selected from the group comprising sensory ingredients, functional ingredients or a combination thereof;
e) Optionally adding promotionals and/or emotionals or other minor ingredients;
   wherein steps c, d, and e may be performed simultaneously or sequentially in any order.

The preferred order of steps is first (c), then (d) and (e).

The invention further relates to a base composition suitable for use in this process.

### DETAILED DESCRIPTION

The invention provides a method for preparing a personal care composition, preferably a cosmetic composition such as a skin cream. Such compositions include but are not limited to skin creams, lotions, vanishing creams, shampoos and the like.

Preferably the composition can be easily spread on the skin. A preferred composition is characterized by an apparent viscosity of from 0.1 to 10000 Pa.s at a shear rate of 10 s⁻¹ to 100 s⁻¹, preferably from 0.1 to 100 Pa.s, more preferred from 0.1 to 20 Pa.s at a shear rate of 10 s⁻¹ or an apparent yield stress of from 0.1 to 10000 Pa, preferably from 1 to 10000 Pa, more preferred from 5 to 1000 Pa, even more preferred from 10 to 100 Pa.

In the context of the invention, a crystallization temperature or phase transition temperature may be determined by using DSC techniques.

In the process for the preparation of a personal care composition, in a first step (a) a base composition comprising from 1 to 90 wt % water and a specific non-ionic surfactant mixture, is provided at a temperature of from 60 to 150° C.

This base composition may be prepared in any suitable way e.g. by mixing surfactant with water and subsequently heating this mixture to a temperature from 60 to 150° C. Alternatively (part) of the ingredients of this concentrate composition are pre-heated to the desired temperature before setting the temperature of the mixture to the desired temperature of between 60 and 150° C.

The temperature of the composition provided in step (a) is from 60 to 150° C. Within this range the temperature is preferably chosen such that it is above the crystallization temperature of the mixture provided. Generally a suitable temperature is from 70 to 90° C.

It is preferred that the base composition is mixed such that a homogeneously mixed composition is provided in step (a). The base composition is preferably in liquid or pasty form.

The base composition comprises a non-ionic surfactant mixture which comprises a first non-ionic surfactant selected from the group consisting of long chain fatty alcohols and long chain fatty acid mono esters of ethylene glycol, -glycerin or -sorbitol or a combination thereof and a second non-ionic surfactant, selected from the group consisting of polyethylene glycol esters of long chain fatty acid, polyethylene glycol ethers of long chain fatty alcohol, or a mixture thereof, wherein the molar ratio of the first non-ionic surfactant to the second non-ionic surfactant is from 10 to 1 to 80 to 1.

The first non-ionic surfactant is preferably selected from a low HLB non-ionic surfactant group comprising long chain fatty alcohol, ethylene glycol, glycerin and sorbitol mono ester of long chain fatty acid.

The second non-ionic surfactant is preferably chosen from high HLB non-ionic surfactants which comprise a bulky hydrophyllic group. The most preferred high HLB non-ionic surfactant is selected from the group comprising polyethylene glycol (PEG) with a polymerization degree between about 20 and about 200 or mixtures thereof. The most preferred degree of polymerization is from 40 to 100.

The base composition is based on the presence of a specific mixture of non-ionic surfactants that provides the desired rheological properties on combination with water. Long chain fatty acids may be included in the base composition. It is important that fatty acids when present are present in their non neutralised form. Therefore when fatty acids are included in the composition their degree of neutralisation is at most 5%, preferably at most 8%, most preferred from 0 to 1%.

Fatty acids may be neutralised by the other ingredients such as a neutralising base. To avoid neutralisation of the fatty acids, the base composition comprises less than 0.1 wt % of neutralising base. Examples of neutralising base are potassium hydroxide, sodium hydroxide and triethanol amine.

The molar ratio of the first non-ionic surfactant to the second non-ionic surfactant is from 10:1 to 80 to 1. In preferred compositions the molar ratio between these two surfactants is preferably from about 25 to 1 to 60 to 1.

The base composition comprises from 10 to 99 wt % of non-ionic surfactant mixture, preferably from 15 to 99%, more preferred from 20 to 80 wt %, most preferred from 40 to 70 wt % non-ionic surfactant mixture on total weight of the base composition.

In a further step (b) the composition obtained in step (a) is cooled to a temperature below 60° C., preferably to a temperature from 5 to 30° C., more preferred from 10 to 25° C. Without wishing to be bound by any theory it is believed that this cooling serves to obtain crystallization of the surfactants in the base composition. The cooling is carried out under conditions whereby shear is avoided especially when the temperature is brought below the crystallization temperature of (any of) the ingredients present in the base composition. Therefore in a preferred embodiment, the invention relates to a process whereby below a temperature of 65° C., preferably below 60° C., the cooling is carried out quiescently.

In the context of the invention quiescent is defined as without substantial shear. This implies that some shear (e.g. due to movement of the container or tube in which the cooling is carried out, or by stirring in a vessel at a rate per minute below 1,) is tolerable. Preferably the cooling is carried out quiescent over the whole temperature range over which the composition is cooled. It is preferred that quiescent cooling is at least applied below a temperature of 60° C. to avoid interference of shear during crystallization of (part of) the ingredients of the concentrate composition.

The cooling is preferably carried out at a rate of from 0.1 to 20° C. per minute, preferably from 0.5 to 3° C./minute.

Optionally the base composition in step (a) is provided with other ingredients such as waxes (e.g. petrolatum) or clays. It is preferred to include high melting ingredients in step (a) where the temperature is still relatively high because generally such ingredients may not distribute homogeneously once temperatures are low, e.g below 30° C., after step (b).

In a further step (c) the base composition is diluted until a non-ionic surfactant concentration between 8 and 30 wt %, preferably 12 and 20 wt %, on total product weight is obtained. The total product weight is the weight of the final personal care product.

It was found that the selection of the final surfactant concentration by choosing a suitable degree of dilution enables the production of products with a varying degree of texture/structure. Generally the higher the degree of dilution, the softer, more flowable the products will be. It will be appreciated that the dilution step is an essential step in the process of the invention. Hence in an exemplary embodiment, when the starting concentrate is at a non-ionic surfactant concentration of 12-30 wt %, the dilution is such that the concentration of surfactant in the final personal care product is lower than that in the concentrate, preferably between 8 and 30 wt %.

In a preferred embodiment, the personal care composition is a cream or a lotion and the dilution in step (c) is to from 12 to 20 wt % non-ionic surfactant.

The dilution in step (c) may be carried out in any suitable apparatus. It is preferred that minimum shear is used to obtain a homogeneous dilution product.

Mixing conditions comparable to manual mixing of 100 ml of composition with a spatula were found to be suitable.

Generally the amount of base composition in the final personal care composition is from 8 to 50 wt %.

The dilution in step (c) is preferably with an aqueous composition. Most preferred the aqueous composition that is used for dilution consists essentially of water.

Generally it is preferred that the temperature during the process in step (c), (d) and (e) is from 5 to 95° C., preferably 10 to 90° C., more preferably from 10 to 40° C., most preferred 20 to 40° C.

The dilution in step (c) is preferably carried out at a temperature of the base composition of from 5 to 95° C. and a temperature of the added aqueous phase for dilution at from 5 to 95° C. It is preferred that the temperature of the added aqueous phase and the base composition in step (c) are both below 60° C., preferably from 10 to 40° C., more preferred 15 to 40° C., most preferred around room temperature.

One of the main advantages of the present process is that it allows the use of one unique base composition for the preparation of a large variety of products that may differ in sensory properties. This wide variation is obtained by on the one hand the dilution step which results in a variety of textures, depending on the dilution factor that is chosen, and on the other hand on the step wise addition of sensory/functional ingredients and promotionals/emotionals and other ingredients.

To obtain the desired variation for customization of products in step (d) the sensory perception and the functionality of the composition are set by adding an ingredient selected from the group comprising sensory ingredients, functional ingredients or a combination thereof.

Functionality of the composition, in the context of the invention, is defined as having an effect on moisturisation degree, sun protection factor, water resistance or a combination thereof. The primary functionality of personal care products according to the invention is generally in their moisturisation capacity. Other functionalities are anti-ageing and effect on firmness.

Generally the functionality of a personal care composition may be controlled by the level and variation of emollients and humectants.

Examples of humectants are glycerol(glycerine), oils, and other alcohols. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. The amount of humectant may range anywhere from 0.5 to 30%, preferably between 1 and 15% by weight of the final personal care composition.

Emollients are typically oil based and in liquid form and may be selected from the group comprising hydrocarbons, silicones, synthetic esters or vegetable esters or their combinations.

Silicone oils may be divided into the volatile and non-volatile variety. The term “volatile” as used herein refers to those materials, which have a measurable vapour pressure at ambient temperature. Volatile silicone oils are preferably chosen from cyclic or linear polydimethyl siloxanes containing from 3 to 9, preferably from 4 to 5 silicon atoms. Linear volatile silicone materials generally have viscosities less than about 5 centistokes at 25° C., whilst cyclic materials typically have viscosities of less than about 10 centistokes.

Non-volatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile silicone emollients useful in the present compositions are the polydimethyl siloxanes having viscosities from about 10 to about 400 centistokes at 25° C.

Among the esters are:
(1) Alkenyl or alkyl esters of fatty acids having 10 to 20 carbon atoms. Examples thereof include isoarachidyl neopentanoate, isononyl isononanoate, oleyl myristate, oleyl stearate, and oleyl oleate
(2) Wax esters such as beeswax, spermaceti, myristyl myristate, stearyl stearate and arachidyl behenate.
(3) Sterol esters of which cholesterol fatty acid esters are examples thereof.

Preferred emollients are selected from the group comprising isoparraffins, mineral oil, cyclic or linear polydimethylsiloxanes, vegetable based oils such as sunflower oil and olive oil or a combination of any of these.

Sensory properties relate to sensorial attributes of the product perceived by the user and may include: smoothness, roughness, ease of rub-in, greasiness, thickness, dragginess, silkiness, velvety, stickiness. Such properties may be controlled via addition of ingredients known to give a sensorial response. In particular, the inclusion of polymeric ingredients such as Simugel™, Sepigel™ and midified starches, has been specifically found to control the perception of silkiness. Silicon oils are found to give a smooth feeling product. High levels of glycerol and thickeners such as Carbopol™ give ‘sticky’ products. High levels of vegetable oils tend to give a greasy product. The level and ingredient type may be varied to give a desired or satisfactorial sensorial response. Thickeners and/or viscosifiers are optionally included to alter the consistency and thickness of the composition and those used include biopolymers, polymers, starches, clays and combinations. The silking agents such as Simugel™, Sepigel™, and starches, may also be used to thicken the composition.

Any combination of the above ingredients may be used to obtain the desired list of sensorical properties.

While most ingredients specifically influence sensorical properties or functional properties, some of the known ingredients will influence both the functional and sensorical characteristics of a composition. The inclusion of such ingredients is preferred.

Therefore in a preferred embodiment, in step (d) the functional and sensorical properties of the composition are set by inclusion of ingredients preferably selected from the group comprising oils (vegetable oils, silicon oils), polymers, and glycerol or a combination thereof.

In a most preferred embodiment, in step (d) the polymers are selected from the group comprising Sepigel™, Simugel™, polyacrylamide, polyacrylates, silicon polymers, siloxane, modified starches or a combination thereof.

If the process includes a step wherein oil and glycerol are added to the composition, these are preferably added separately, one after the other without a preference for a specific order.

In one embodiment, the functional ingredients in step (d) may beneficially comprise little additional thickener. Suitable thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Alternative thickener is clay. Amounts of the thickener may range from 0.0001 to 2%, usually from 0.001 to 1%, by weight of the personal care composition, if at all.

Optionally, in a further step (e) promotionals, emotionals, actives, or other minor ingredients may be added. Actives are defined as skin benefit agents other than emollients and other than ingredients that merely improve the physical characteristics of the composition. Although not limited to this category, general examples include additional anti-sebum ingredients such as talcs and silicas, and sunscreens. Further examples include silk protein, fragrances, colouring agents, healthy skin ingredients such as AHA, collagen, amino acids; vitamins such as vitamin A and vitamin E, triple lipids such as lecithin, soy sterol; or combinations thereof.

Another minor ingredient for optional addition in step (e) according to the invention may be a retinoid. Retinoids increase collagen synthesis by dermal fibroblasts. This results in protection from sun damage and smoothening of wrinkled skin. The term “retinoids” as used herein, includes retinoic acid, retinol, retinal, and retinyl esters. Included in the term “retinoic acid” are 13-cis retinoic acid and all-trans retinoic acid.

The term “retinol” as used herein includes the following isomers of retinol: all-trans-retinol, 13-cis-retinol; 11-cis-retinol, 9-cis-retinol, 3,4-didehydro-retinol. Preferred isomers are all-trans-retinol, 13-cis-retinol, 3,4-didehydro-retinol, 9-cis-retinol. Most preferred is all-trans-retinol, due to its wide commercial activity.

Retinyl ester is an ester of retinol. The term “retinol” has been defined above. Retinyl esters suitable for use in the present invention are C₁-C₃₀ esters of retinol, preferably C₂-C₂₀ esters, and most preferably C₂, C₃, and C₁₆ esters because they are more commonly available.

If present, the retinoids in the present invention may be present in an amount of from 0.001% to 10%, preferably from 0.01% to 1%, and most preferably from 0.01% to 0.05% by weight of the final personal care composition.

For all above identified ingredients that may be added in step (d) or (e), it is optional that the ingredients are present in the form of pre-mixes before they are added. For example minor ingredients may be pre-mixed with water before they are included, to facilitate their dosing.

Alternatively the above-identified ingredients are added as such or mixed with other ingredients before addition.

In a preferred embodiment of the process according to the invention the base composition is a flowable composition. Also a pumpable paste may suitably be used as a base composition for step (a). It is preferred that all further ingredients that are added are flowable/pumpable as well rather than being powders that need special handling. In the event that the use of powders may not be avoided, they are preferably added as dispersion in water or oil.

The process according to the invention may be carried out in a central manufacture set up such as a factory line. The process is suitable for application on a large, industrial scale because it starts from a suitable base composition, followed by processing and addition of ingredients such that individual variants result.

According to an alternative embodiment the process is carried out at smaller scale in an apparatus made suitable therefore.

The process is especially suitable for use on such small scale. Such apparatus may be placed in a shop, a distribution centre, small factory outlet or other retail outlet and would enable the consumer to select the specific variety needed. From a base composition the final product is prepared by use of the process according to the invention.

In a further aspect the invention relates to use of a process according to the invention to provide a wide variety of personal care compositions which vary in sensorial and functional characteristics.

The process according to the invention is also suitable for use in controlling the sensorial and functional properties of a personal care composition.

In an even further embodiment, the base composition may be sold as such and used to prepare individual products at small scale. In this embodiment, an individual consumer may use the base composition to prepare her individual personal care product at home in the amount desired and with added ingredient according to her own needs. Alternatively the base composition is used in an apparatus wherein further steps (c-e) are carried out.

Therefore the invention in another aspect relates to a process for preparing a base composition suitable for use in preparing a personal care composition, the process comprising the steps of
a) providing a composition comprising 1 to 90 wt % water and a non-ionic surfactant mixture which comprises a first non-ionic surfactant selected from the group consisting of long chain fatty alcohols and long chain fatty acid mono esters of -ethylene glycol, -glycerin, -sorbitol or a combination thereof and a second non-ionic surfactant, selected from the group consisting of polyethylene glycol esters of long chain fatty acid, polyethylene glycol ethers of long chain fatty alcohol, or a mixture thereof,
   wherein the molar ratio of the first non-ionic surfactant to the second non-ionic surfactant is from 10 to 1 to 80 to 1;
b) heating the composition to a temperature from 60 to 150° C., preferably to a temperature above the crystallization temperature of the composition, most preferred to a temperature from 70 to 90° C.;
c) Cooling to a temperature below 60° C., preferably to a temperature from 10 to 30°;
d) Packing the composition

In another aspect the invention relates to a base composition which is obtained by this process. The obtained composition is suitable for use in preparing a personal care composition in a process where water is added to the base composition. The obtained base composition differs from compositions which are a mere admixture of a non-ionic surfactant mixture and water. Without wishing to be bound by any theory it is believed that the obtained base composition is in such physical state that it can easily take up water and form a personal care composition with pourable or sqeeuzable consistency.

It is preferred that the base composition is prepared in a process consisting of the above steps (a-d). It is also preferred that the process for preparing the base composition does not contain a step wherein water is removed from the concentrate.

The base composition may be supplemented with further ingredients such as ingredients suitable for setting the sensory perception and functionality of a personal care composition, promotionals, emotionals, minor ingredients or combinations thereof. These ingredients are specified further above. Water removal may lead to an undesired change in physical properties, especially on dilution of the base composition.

The invention is now illustrated by the following non-limiting examples.

### EXAMPLES

### Example 1

### Formation of a Range of Products from a Single Base Concentrate

An aqueous concentrate composition was prepared by mixing the surfactants specified in table 1 with water, and heating the mixture to 80° C. The final non-ionic surfactant concentration in the base composition was 15 wt % non-ionic surfactant on total weight of the base composition. The remainder was water. The concentrate was prepared by mixing with an anchor propellor at a rotation rate of around 50 rpm in a 1 litre jacketed beaker for 10 minutes. The concentrate was cooled to 25° C. at 1° C./min under quiescent conditions; i.e. without mixing. Subsequently the concentrate was diluted with water to a concentration of 5% surfactant on total product to a volume of 100 mL by manually mixing with a spatula until a homogeneous mixture was obtained. Veegum™ (inorganic, complex, colloidal magnesium aluminum silicate) and Keltrol™ (xanthan gum) were added to this diluted base composition in an amount of 1 wt % each, by stirring.

TABLE 1
Proportion of surfactant mixture.

| | Weight fraction relative to |
|---|---|
| Ingredient | total surfactants (wt %) |
| Stearic acid | 24 |
| Glyceryl dilaurate | 12 |
| Sorbitan stearate | 27 |
| Cetearyl Alcohol | 24 |
| PEG 100 stearate | 12 |

The obtained product was a white viscose cream.

TABLE 2
Proportion of surfactant mixture Example 2

| Surfactants | Weight ratio |
|---|---|
| Cetyl alcohol | 50 |
| Glycerin monostearate | 30 |
| PEG 100 stearate | 20 |

Preparation method of example 2 is the same as example 1, but the surfactant concentration is 90% on the total weight of base composition. The remainder was water. A solid and waxy material is obtained after cooling. This base composition may be used as such or may be admixed with further ingredients to obtain a personal care composition.

1. Process for the preparation of a personal care composition, which process comprises the steps of
a) Providing at a temperature of from 60 to 150° C., a base composition comprising 1 to 90 wt % water and a non-ionic surfactant mixture which comprises a first non-ionic surfactant selected from the group consisting of long chain fatty alcohols and long chain fatty acid mono esters of ethylene glycol, -glycerin, -sorbitol or a combination thereof and a second non-ionic surfactant, selected from the group consisting of polyethylene glycol esters of long chain fatty acid, polyethylene glycol ethers of long chain fatty alcohol, or a mixture thereof, wherein the molar ratio of the first non-ionic surfactant to the second non-ionic surfactant is from 10 to 1 to 80 to 1;
b) Cooling the composition obtained in step (a) to a temperature below 60° C.;
c) diluting the base composition until a surfactant concentration between 8 wt % and 30 wt % is obtained;
d) setting the sensory perception and the functionality of the composition by adding at least one ingredient selected from the group comprising sensory ingredients, functional ingredients or a combination thereof;
e) Optionally adding promotionals and/or emotionals or other minor ingredients;
wherein steps c, d, and e may be performed simultaneously or sequentially in any order.

2. Process according to claim 1 wherein the base composition comprises less than 1 wt % of ionic surfactant.

3. Process according to claim 1 wherein the molar ratio between the first and second surfactant is from 25 to 1 to 60 to 1.

4. Process according to claim 1 wherein the second non-ionic surfactant is selected from polyethylene glycol (PEG)esters of long chain fatty acid or ethers of long chain alcohol with a polymerization degree between about 20 and about 200 or mixtures thereof.

5. Process according to claim 1 wherein the first non-ionic surfactant is selected from the group comprising long chain fatty alcohol, and long chain fatty acid mono ester of glycerine, ethylene glycol, and sorbitol or a combination thereof.

6. Process for preparing a base composition suitable for use in the preparation of a personal care composition, the process comprising the steps of
a) providing a composition comprising from 1 to 90 wt % water and a non-ionic surfactant mixture which comprises a first non-ionic surfactant selected from the group consisting of long chain fatty alcohols and long chain fatty acid mono esters of -ethylene glycol, -glycerin, -sorbitol or a combination thereof and a second non-ionic surfactant, selected from the group consisting of polyethylene glycol esters of long chain fatty acid, polyethylene glycol ethers of long chain fatty alcohol, or a mixture thereof, wherein the molar ratio of the first non-ionic surfactant to the second non-ionic surfactant is from 10 to 1 to 80 to 1;
b) heating the composition to a temperature from 60 to 150° C., preferably to a temperature above the crystallization temperature of the composition
c) Cooling to a temperature below 60° C.,
d) Packing the composition.

7. Base composition obtainable by the process according to claim 6.
